(19) United States
(12) Patent Application Publication
(10) Pub. No.: US 2026/0279592 A1
(43) Pub. Date: Sep. 17, 2026

(54) SYSTEMS AND METHODS FOR AN ARTIFICIAL INTELLIGENCE HEALTH COACH WITH BIOMETRIC DATA INTEGRATION AND PERSONALIZED HEALTH INTERVENTION GENERATION

(71) Applicant: Joseph MERCOLA, Ormond Beach, FL (US)

(72) Inventor: Joseph MERCOLA, Ormond Beach, FL (US)

(21) Appl. No.: 19/565,963

(22) Filed: Mar. 13, 2026

Related U.S. Application Data
(60) Provisional application No. 63/771,966, filed on Mar. 14, 2025.

(51) Int. Cl. G16H 50/30; A61B 5/00; A61B 5/145; G09B 5/02; G09B 5/04; G10L 13/033; G10L 15/18; G10L 15/183; G10L 15/22; G10L 25/63

(52) U.S. Cl. CPC G16H 50/30; A61B 5/14532; A61B 5/4803; A61B 5/7275; G09B 5/02; G09B 5/04; G10L 13/033; G10L 15/1807; G10L 15/183; G10L 15/22; G10L 25/63

(57) ABSTRACT

A method uses an artificial intelligence health coach system that includes receiving a voice input from a user, interpreting the voice input with natural language processing performed by an artificial intelligence model, which includes a large language model, and receiving a glucose level from a continuous glucose monitor. The method further includes receiving an insulin level from a database, calculating a Homeostatic Model Assessment of Insulin Resistance score from the glucose level and the insulin level, comparing the score to historical scores associated with the user, and identifying a predicted risk of a hyperglycemic episode. The method also includes generating a preventative health recommendation modified based on whether the score has changed over time, requesting additional health information from the user, modifying the recommendation based on the additional information, and outputting the modified recommendation to the user. The method maintains persistent user data as contextual memory across interactions.

### CROSS-REFERENCE TO RELATED APPLICATIONS

This application claims priority to and the benefit of U.S. Provisional Application No. 63/771,966, filed on Mar. 14, 2025, and titled “Systems and Methods for a Health Coach”, the contents of which are hereby incorporated herein by reference in their entirety.

### FIELD OF THE INVENTION

The present invention relates generally to systems and methods for a health assistant, and more specifically, to a health assistant that uses generative artificial intelligence (“AI”) modules for generating health-assistance outputs.

### BACKGROUND OF THE INVENTION

Medical information is increasingly accessible to ordinary individuals through public means such as the internet. However, gathering and identifying relevant medical information remains a challenge to untrained individuals even in situations where the relevant medical information is not complicated. People increasingly prefer not to visit a medical professional to obtain basic medical information. The increased cost of medical care, even for basic interactions that involve no treatment, discourages individuals from seeking answers to health-related questions from practitioners. Individuals increasingly turn to the internet to find information for themselves, yet the internet provides unfiltered information without personalization to the individual's specific health circumstances or current physiological state.

Accordingly, there exists a need for a health coach system that combines advanced natural language processing, real-time biometric data integration from wearable and medical devices, physiological trend analysis, metabolic index calculation, historical health data comparison, and personalized health intervention generation within a unified artificial intelligence platform capable of interacting with users through natural voice conversation while maintaining consistent personality characteristics and contextual memory across interactions.

The present disclosure provides a solution for these and other problems.

### SUMMARY OF THE INVENTION

The term embodiment and like terms, e.g., implementation, configuration, aspect, example, and option, are intended to refer broadly to all of the subject matter of this disclosure and the claims below. Statements containing these terms should be understood not to limit the subject matter described herein or to limit the meaning or scope of the claims below. Embodiments of the present disclosure covered herein are defined by the claims below, not this summary. This summary is a high-level overview of various aspects of the disclosure and introduces some of the concepts that are further described in the Detailed Description section below. This summary is not intended to identify key or essential features of the claimed subject matter. This summary is also not intended to be used in isolation to determine the scope of the claimed subject matter. The subject matter should be understood by reference to appropriate portions of the entire specification of this disclosure, any or all drawings, and each claim.

The present disclosure relates generally to systems and methods for a health assistant and more specifically to a health assistant that uses a generative artificial intelligence large language model to analyze biometric data and medical information and to provide personalized health information to a user through voice and other outputs. According to some implementations, health information systems provide health-related data or responses to health queries. Some implementations integrate real-time biometric data from wearable sensors and medical devices with conversational artificial intelligence. For example, some implementations receive continuous biometric data streams, analyze physiological trends from that data, calculate indices such as insulin resistance scores, compare current biomarker values against historical baselines for the same individual, and synthesize this information into health intervention recommendations delivered through natural voice conversation while maintaining persistent user profiles that combine conversation history, emotional state data, biometric trends, and personal preferences.

According to certain aspects of the present disclosure, a method is directed to providing personalized health interventions using an artificial intelligence health coach. The method includes receiving a voice input from a user and interpreting it with natural language processing to determine its meaning, including whether the voice input requests personalized health intervention information. The method includes receiving a glucose level from a continuous glucose monitor and an insulin level retrieved from a database. The method includes calculating, with a processor, a Homeostatic Model Assessment of Insulin Resistance score from the glucose level and the insulin level, comparing the score to historical scores associated with the user to determine whether the score has changed over time, and identifying a predicted risk of a hyperglycemic episode based on the comparison. The method includes generating, using an artificial intelligence model comprising a large language model, a preventative health recommendation based on the predicted risk and modified based on whether the score has changed over time, and outputting the preventative health recommendation to the user through voice, visual, or other output modalities. The method further includes iteratively refining the health recommendations through additional user inputs and updated biometric data over time.

According to other aspects of the present disclosure, a health coach system maintains a persistent user data profile combining conversation history, emotional and contextual data, biometric data trends, and personal preferences, and uses this profile to progressively improve the accuracy and personalization of health guidance across multiple interactions while maintaining consistent personality traits and a coherent conversational identity.

According to other aspects of the present disclosure, a non-transitory computer-readable medium stores instructions that, when executed by one or more processors, cause a device to perform the methods described herein.

According to other aspects of the present disclosure, a method includes receiving, from a user, a voice input through a voice interface. The method further includes interpreting the voice input with natural language processing performed by an artificial intelligence model that includes a large language model to determine a meaning of the voice input, the large language model including whether the voice input requests personalized health intervention information. The method further includes receiving a glucose level from a continuous glucose monitor, receiving an insulin level retrieved from a database, and calculating, with a processor, a Homeostatic Model Assessment of Insulin Resistance score from the glucose level and the insulin level. The method further includes comparing the Homeostatic Model Assessment of Insulin Resistance score to historical Homeostatic Model Assessment of Insulin Resistance scores associated with the user to determine whether the Homeostatic Model Assessment of Insulin Resistance score has changed over time. The method further includes identifying, based on the Homeostatic Model Assessment of Insulin Resistance score and the comparison to historical scores, a predicted risk of a hyperglycemic episode. The method further includes generating, with the processor using the artificial intelligence model, a preventative health recommendation based on the predicted risk of the hyperglycemic episode and modified based on whether the Homeostatic Model Assessment of Insulin Resistance score has changed over time. The method further includes requesting, with a voice output through the voice interface, additional health information from the user. The method further includes receiving the additional health information from the user as a voice input. The method further includes modifying the preventative health recommendation based on the additional health information. The method further includes outputting the modified preventative health recommendation to the user.

According to other aspects of the present disclosure, a non-transitory computer-readable medium stores instructions that, when executed by one or more processors of a device, cause the device to receive a user input, and interpret the user input with natural language processing performed by an artificial intelligence model that includes a large language model to determine a meaning of the user input, including whether the user input requests personalized health intervention information. The instructions further cause the device to receive a glucose level from a continuous glucose monitor, and to receive an insulin level retrieved from a database; calculate a metabolic health index from the glucose level and the insulin level, the metabolic health index including at least an insulin resistance index calculated from the glucose level and the insulin level. The instructions further cause the device to analyze the glucose level, the insulin level, and the metabolic health index with the artificial intelligence model, and to identify, based on the metabolic health index and the meaning of the user input, a physiological trend of the user. The instructions further cause the device to compare the metabolic health index to historical metabolic health index data associated with the user to determine whether the metabolic health index has changed over time. The instructions further cause the device to generate, using the artificial intelligence model, personalized health intervention information relating to the identified physiological trend of the user and modified based on whether the metabolic health index has changed over time. The instructions further cause the device to output the personalized health intervention information to the user.

According to other aspects of the present disclosure, a health coach system includes a voice interface configured to receive a user input including at least one of a spoken input through a microphone, a typed input through a keyboard, or a gestural input through a camera, the voice interface including computer vision models configured to (a) recognize and interpret gestures and facial expressions, and (b) to process the user input using natural language processing. The user input includes the spoken input the voice interface performs prosody analysis on the spoken input to detect emotional cues. The health coach system further includes one or more sensors including a continuous glucose monitor configured to provide a glucose level to the health coach system, and one or more data sources configured to provide an insulin level retrieved from a database. The health coach system further includes one or more processors configured to calculate a Homeostatic Model Assessment of Insulin Resistance score from the glucose level and the insulin level, the one or more processors further being configured to compare the Homeostatic Model Assessment of Insulin Resistance score to historical Homeostatic Model Assessment of Insulin Resistance scores associated with the user to determine whether the score has changed over time. The health coach system further includes a large language model configured to receive the processed user input, the biometric data, and the calculated Homeostatic Model Assessment of Insulin Resistance score, the large language model being further configured to analyze the biometric data and identify a physiological trend or condition of the user, the large language model being further configured to generate personalized health intervention information relating to the identified physiological trend or condition and modified based on whether the Homeostatic Model Assessment of Insulin Resistance score has changed over time. The health coach system further includes a user data storage configured to store conversation records, recorded emotional states, biometric data history, and user preferences as a persistent user profile. The health coach system further includes an output interface configured to output the personalized health intervention information to the user through at least one of a speaker or a screen. The health coach system uses the persistent user profile as contextual memory to interpret user inputs and generate personalized health intervention information across multiple interaction sessions.

The above summary is not intended to represent each embodiment or every aspect of the present disclosure. Rather, the foregoing summary merely provides an example of some of the novel aspects and features set forth herein. The above features and advantages, and other features and advantages of the present disclosure, will be readily apparent from the following detailed description of representative embodiments and modes for carrying out the present invention, when taken in connection with the accompanying drawings and the appended claims. Additional aspects of the disclosure will be apparent to those of ordinary skill in the art in view of the detailed description of various embodiments, which is made with reference to the drawings, a brief description of which is provided below.

### BRIEF DESCRIPTION OF THE DRAWINGS

The disclosure, and its advantages and drawings, will be better understood from the following description of representative embodiments together with reference to the accompanying drawings. These drawings depict only representative embodiments and are therefore not to be considered as limitations on the scope of the various embodiments or claims.

FIG. 1 is a diagrammatic illustration representing a technical environment for a health coach system, in accordance with an exemplary embodiment of the present disclosure.

FIG. 2 is a diagrammatic illustration representing components of the health coach system of FIG. 1, in accordance with an exemplary embodiment of the present disclosure.

FIG. 3 is a flowchart illustrating a method that uses the health coach system of FIG. 1, in accordance with an exemplary embodiment of the present disclosure.

### DETAILED DESCRIPTION

Various embodiments are described with reference to the attached figures, where like reference numerals are used throughout the figures to designate similar or equivalent elements. The figures are not necessarily drawn to scale and are provided merely to illustrate aspects and features of the present disclosure. Numerous specific details, relationships, and methods are set forth to provide a full understanding of certain aspects and features of the present disclosure, although one having ordinary skill in the relevant art will recognize that these aspects and features can be practiced without one or more of the specific details, with other relationships, or with other methods. In some instances, well-known structures or operations are not shown in detail for illustrative purposes. The various embodiments disclosed herein are not necessarily limited by the illustrated ordering of acts or events, as some acts may occur in different orders and/or concurrently with other acts or events. Furthermore, not all illustrated acts or events are necessarily required to implement certain aspects and features of the present disclosure.

For purposes of the present detailed description, unless specifically disclaimed, and where appropriate, the singular includes the plural and vice versa. The word “including” means “including without limitation.” Moreover, words of approximation, such as “about,” “almost,” “substantially,” “approximately,” and the like, can be used herein to mean “at,” “near,” “nearly at,” “within 3-5% of,” “within acceptable manufacturing tolerances of,” or any logical combination thereof. Similarly, terms “vertical” or “horizontal” are intended to additionally include “within 3-5% of” a vertical or horizontal orientation, respectively. Additionally, words of direction, such as “top,” “bottom,” “left,” “right,” “above,” and “below” are intended to relate to the equivalent direction as depicted in a reference illustration; as understood contextually from the object(s) or element(s) being referenced, such as from a commonly used position for the object(s) or element(s); or as otherwise described herein.

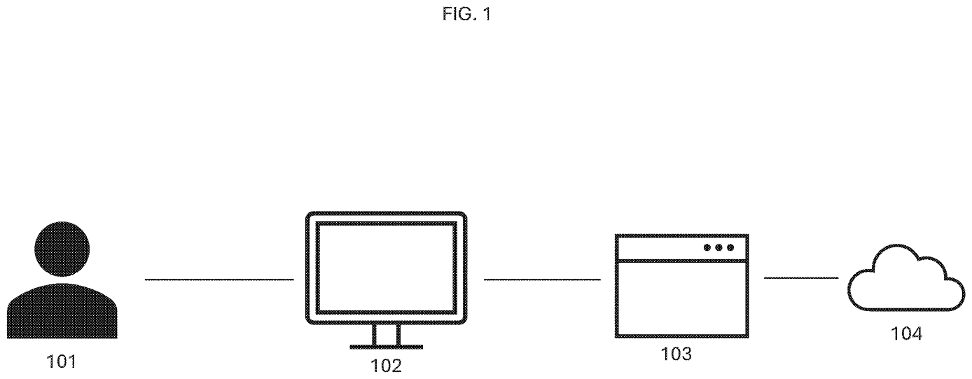

Referring now to FIG. 1, an exemplary technical environment (100) is illustrated in which the artificial intelligence health coach may be used, showing a user (101), a device (102), a web browser or application interface (103), and a cloud-hosted health coach platform (104), consistent with the disclosed embodiments. The user (101) is a person or entity that uses a device (102) on which the health coach is installed or running. The device (102) includes a non-transitory computer-readable medium that stores data and a processor configured to execute health coach software. The device (102) may be any device capable of running the health coach software, including but not limited to a cell phone, a laptop computer, a tablet computer, a desktop computer, a smart speaker, a dedicated health monitoring device, or any other computing device with input and output capabilities.

The health coach operates, optionally, locally on the device (102) or may be operated using a web browser (103) or an application or other user interface that is connected to a cloud-hosted health coach platform (104). In cloud-hosted embodiments, the device (102) communicates with the cloud-hosted platform (104) over a network connection. The user (101) interacts with the health coach through a variety of input means on the device (102) to initiate and conduct a conversation with the health coach. The health coach uses a variety of means to create and output responses from the device (102) to the user (101).

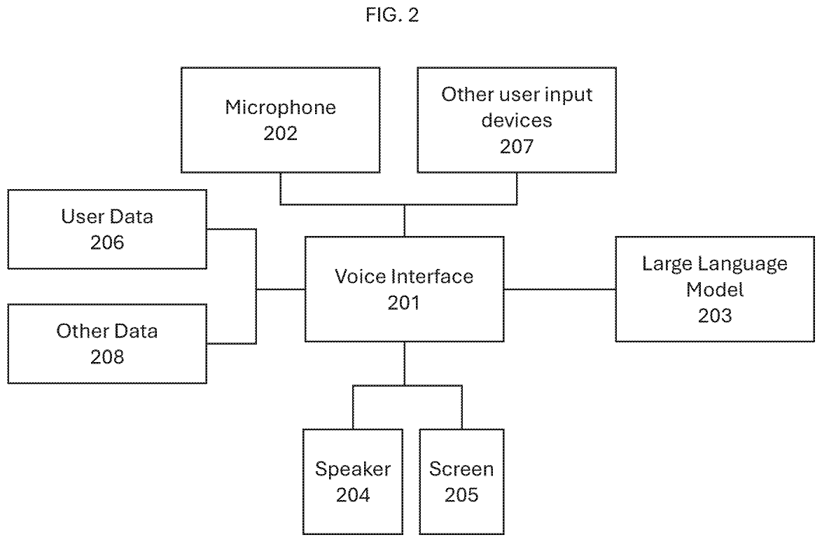

Referring now to FIG. 2, exemplary components of the health coach system (200) include a voice interface (201), a microphone (202), a large language model (203), a speaker (204), a screen (205), user data storage (206), other user input devices (207), and other data sources (208), consistent with the disclosed embodiments. The voice interface (201) serves as the primary interaction layer between the user and the large language model (203), handling both input processing and output generation.

The voice interface (201) is configured to receive spoken inputs from the user through a microphone (202) or other voice input devices. The voice interface (201) performs advanced speech recognition to accurately transcribe the intended meaning of inputs into text, including prosody analysis to detect emotional cues. For example, the voice interface (201) may detect pitch variation indicating stress, a trembling voice suggesting anxiety, or other vocal characteristics indicative of the user's emotional state, and may adjust responses based on the detected emotional state. The voice interface (201) is able to disambiguate between two or more intended meanings of an input, process complex or multi-part questions or inputs with multiple intents, and understand context-dependent queries based on the conversation history. For example, the voice interface (201) may process a complex input such as “I feel tired and my sugar's high, what should I do?” by identifying both the subjective symptom report and the biometric data reference and determining that the user is requesting personalized health intervention information.

The voice interface (201) is configured to transform the spoken input into a form that the large language model (203) can process, such as a text input. The voice interface (201) is configured to correct and clarify input mistakes including typos, incomplete inputs, and other input errors. The voice interface (201) may translate abbreviations or numerical inputs into forms that the large language model (203) can process more easily or more accurately, such as translating an input of several numbers into words.

The voice interface (201) is also configured to receive other inputs that are not spoken through other user input devices (207). The other user input devices (207) include input devices configured to receive active user inputs such as a keyboard configured to receive typed messages or a camera configured to detect gestures, as well as input devices configured to receive passive user inputs such as a camera configured to detect facial expressions. The other input devices (207) also include a wearable health monitoring device, a fitness tracker, a smart medical device, a continuous glucose monitor, or any other device or component that can provide biometric data to the health coach. The voice interface (201) includes computer vision models to recognize and interpret gestures and facial expressions. The voice interface (201) analyzes the other inputs received from the other user input devices (207) and transforms the other inputs into a form that the large language model (203) can process.

The voice interface (201) optionally combines a plurality of inputs from a plurality of input devices to create one combined input to send to the large language model (203). The voice interface (201) may selectively send or not send certain inputs to the large language model (203) based on whether the user would expect to receive a response from the health coach. For instance, if the user nods in response to a question from the health coach, the health coach may need the large language model (203) to generate the next conversation output. If the user rolls their eyes while speaking, the large language model (203) may need that information to generate an appropriate output responding to the spoken input, but the large language model (203) may not need to generate a separate response to the eye roll.

The large language model (203) is configured to receive the input from the voice interface (201), generate a responsive output, and send the responsive output to the voice interface (201). The large language model (203) may be an existing commercially available large language model or a custom large language model. The large language model (203) may be a custom model trained on health-specific datasets including medical literature, anonymized patient records, biometric trend data, scientific papers, case studies, and code repositories to enhance its ability to interpret biometric data and generate health-focused outputs. Training includes cleaning and tokenizing the data and incorporating it into the large language model architecture so the large language model (203) can recognize patterns relating to domain-specific health information. Alternatively, the large language model (203) may be a commercial large language model adapted with fine-tuning for conversational health coaching.

The large language model (203) is configured to recognize diverse phrasings and terminologies that may express the same intent. The large language model (203) uses natural language processing techniques to understand and generate human-like language patterns. The large language model (203) contextually understands user queries, recognizes colloquialisms, idioms, and regional expressions, and interprets and responds to complex multi-part user inputs. The large language model (203) adapts to evolving user language and new expressions over time. The large language model (203) may use humor and casual language when appropriate.

The large language model (203) or the voice interface (201) optionally introduce controlled imperfections into the outputs to create more humanlike output. The controlled imperfections include occasional hesitations, self-corrections, admitting uncertainty or a lack of knowledge, subtle variations in response time to mimic human thought processes, and other controlled imperfections. The voice interface (201) performs text-to-speech synthesis involving emotion modulation on the responsive output generated by the large language model (203) to generate outputs that are humanlike in tone, pacing, and emotional expression.

The voice interface (201) and the large language model (203) are configured to incorporate consistent personality traits and a backstory into the health coach. This includes maintaining a consistent tone across different conversations and across multiple interaction sessions with the same user, and using style transfer techniques to adapt outputs to align with the health coach's defined personality traits and backstory. The health coach maintains a coherent conversational identity such that the user experiences the health coach as a consistent entity across all interactions.

The voice interface (201) is configured to output the responsive output to the user through a speaker (204) as an audio output, to a screen (205) as text or graphical output, or through any other output method or medium. The voice interface (201) is configured to send an output to more than one output medium simultaneously, or to output a single output across multiple output devices. The voice interface (201) determines which output method or methods to use based on the conversational context. Some input and output components perform dual functions, such as a screen providing a user interface that functions as both an input method for the user and an output method for text, images, and other user interface elements.

The voice interface (201) includes conversation steering rules or techniques that reject or modify inputs or outputs in order to keep the user or the health coach from deviating from certain topics or from discussing certain topics. The voice interface (201) includes a turn-taking mechanism to mimic natural dialogue, where the voice interface (201) attempts to respond only when the user has finished providing input. The voice interface (201) incorporates appropriate pauses and filler words into the output to simulate human speech patterns.

The health coach includes user data storage (206) containing previous conversation records, recorded emotional and context data, biometric data history, and other forms of user data. The user data storage (206) may be stored locally on the device (102) or on a cloud server, and the location of user data storage (206) is independent of where the health coach software operates. The health coach is configured to save a conversation state as user data and resume the conversation at a later time. The user data is anonymized and encrypted to provide ethical and privacy protections to the user. The health coach is configured to collect and store data only after receiving the user's informed consent, consistent with applicable data privacy regulations and ethical guidelines.

The health coach uses the user data storage (206) for a variety of purposes. The user data includes personal information such as user demographics and preferences, allowing the health coach to adjust the language and tone of outputs to account for cultural nuances and sensitivities. The user data includes contextual memory where previous parts of the current conversation and prior conversations are recorded and utilized by the health coach to create responsive outputs. The health coach interprets ambiguous voice inputs based on contextual memory, historical inputs, or recorded emotional states. For example, if a user in a previous conversation had clarified that an ambiguous input was their pronunciation of a particular word, the health coach recognizes that word in future conversations without needing clarification. Similarly, if the user has reacted positively or negatively to outputs with certain attributes, the health coach attempts to generate more or fewer outputs with those attributes respectively.

The dynamic nature of a conversation may require the health coach to compare the circumstances under which user data was generated to the present circumstances to determine if the health coach should adjust its operations. By dynamically updating and relying on the user data to adjust the operation of the health coach, the health coach more accurately interprets and responds to user inputs, creating an improved user experience.

The health coach includes other data sources (208) containing data received from external sources such as search engines, and data generated and saved by the health coach that is not user data. The other data (208) is updated frequently to allow the health coach to generate relevant and accurate outputs and responses. The other data (208) may include data from other health coach instances and may include anonymized user data from other users collected with the informed consent of those users.

The health coach performs functions in addition to generating responses and having conversations with the user. The health coach functions as a security or authentication system by using voice analysis to match an input with previous voice inputs to confirm the identity of a user. Inputs may be matched based on user data such as emotional markers and voice characteristics. The health coach helps the user maintain a personal journal by prompting the user for inputs such as specific thoughts and recording those thoughts into a journal, providing additional benefits including analysis of the user's thoughts. The health coach automatically categorizes and analyzes the user's inputs to provide insights or identify trends. The health coach controls connected devices or provides other helpful features and functions to the user.

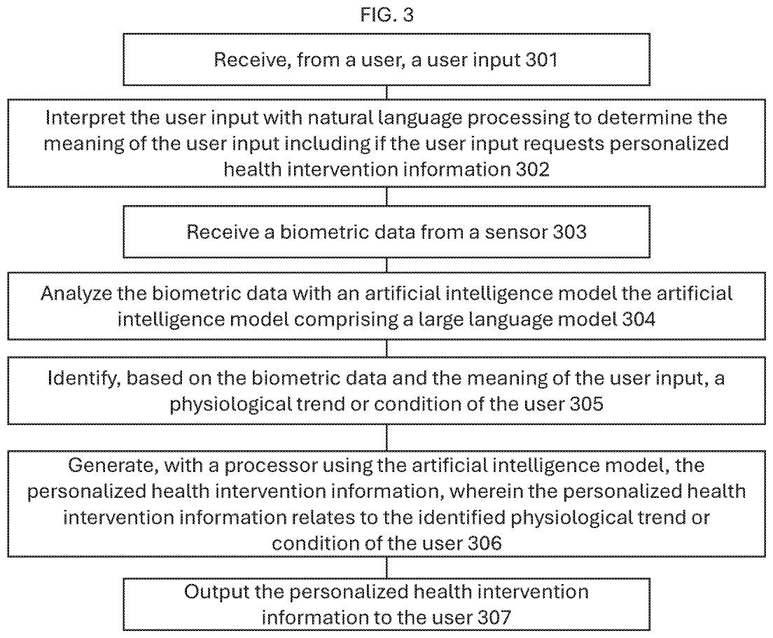

Referring now to FIG. 3, a process for an exemplary operation of the health coach (300) includes receiving a user input (301), interpreting the user input with natural language processing (302), receiving biometric data (303), analyzing the biometric data with an artificial intelligence model (304), identifying a physiological trend or condition (305), generating personalized health intervention information (306), and outputting the personalized health intervention information to the user (307), consistent with the disclosed embodiments.

At step (301), the health coach receives from a user a user input. The user input may be a voice input spoken by the user such as a voice command. The user input may be any other form of input including physical inputs on an input device, gestures, facial expressions, or other inputs.

At step (302), the health coach interprets the user input with natural language processing to determine the meaning of the user input, including whether the user input requests personalized health intervention information. The natural language processing is performed by an artificial intelligence model that includes a large language model. The user input may request personalized health intervention information by requesting a treatment, a health recommendation, medical advice, or information about the user's health condition or symptoms.

At step (303), the health coach receives biometric data. The biometric data is received from a sensor. The sensor may be any sensor including a camera, an input device, a wearable health monitoring device, a fitness tracker, a smart medical device, a continuous glucose monitor, or any other device or component that can provide biometric data to the health coach. The sensor may provide biometric data that the sensor detected and recorded or biometric data that was stored and recorded in the past. The biometric data includes at least one physiological measurement.

The biometric data may include real-time biometric data recorded and immediately transmitted to the health coach. The biometric data may include lab test results that are automatically retrieved by the sensor or by the health coach from a database. The lab data includes data obtained from laboratory, metabolic, hormonal, and microbiome-related tests that provide specific health data including specific biomarker data. For example, the biometric data may include fasting insulin levels, cholesterol levels, HbA1c values, glucose levels, heart rate, heart rate variability, blood oxygen saturation, or other biometric data. The biometric data may include real-time metrics such as glucose from a continuous glucose monitor or heart rate variability from a fitness tracker, or stored data such as HbA1c from lab results retrieved via API from a medical database. The health coach integrates multiple data types to assess trends including circadian rhythm disruptions, metabolic shifts, or other physiological patterns. The biometric data may include multiple physiological measurements integrated to assess a composite health index representing multiple health-related indicators.

At step (304), the health coach analyzes the biometric data with the artificial intelligence model comprising the large language model. The health coach provides the large language model with the user's input and other information including but not limited to information relating to blood sugar patterns, blood glucose variability, the Homeostatic Model Assessment for Insulin Resistance, insulin response, the risk of insulin resistance, diet effects on biometric data, lipid profiles, the risk of metabolic syndrome indicators, and other domain-specific health information. The large language model is trained on domain-specific information related to health and medicine and is further tailored for health analysis as described above. The health coach integrates biometric data by embedding it into its input context, for example “User reports fatigue; glucose: 180 milligrams (mg)/deciliter (dL); insulin: 15 micro-units (μU)/milliliter (mL)” and cross-referencing health knowledge to generate outputs. The health coach may calculate a Homeostatic Model Assessment of Insulin Resistance (HOMA-IR) score from glucose and insulin values.

At step (305), the health coach identifies, based on the biometric data and the meaning of the user input, a physiological trend or condition of the user. The health coach may identify that several symptoms included in the user input or other information accessible to the health coach suggest a particular cause for the user's symptoms. The physiological trend or condition of the user may be a predicted future health outcome. Identification of the physiological trend or condition may include the identification of specific metabolic biomarkers. The physiological trend or condition may include an insulin resistance index and a metabolic health index calculated by the processor from glucose and insulin levels. As used herein, the metabolic health index comprises at least an insulin resistance index calculated from glucose and insulin levels, such as a Homeostatic Model Assessment of Insulin Resistance (HOMA-IR) score.

At step (306), the health coach generates, with a processor using the artificial intelligence model, the personalized health intervention information, wherein the personalized health intervention information relates to the identified physiological trend or condition of the user. The health coach may generate a recommended dietary modification, a supplement recommendation, a lifestyle intervention, a warning about a predicted future adverse health event, or other personalized health intervention information. The health coach may suggest a low-carb meal based on elevated glucose trends or recommend specific actions based on calculated HOMA-IR scores.

At step (307), the health coach outputs the personalized health intervention information to the user. The output may be in the form of a voice output through the speaker (204), a visual output through the screen (205), or through any other output device or combination of output devices.

The health coach performs additional steps including comparing the user's metabolic health index to historical metabolic health index data associated with the user to determine if the user's metabolic health index has changed over time, and modifying the personalized health intervention information based on whether the user's metabolic health index has changed over time before outputting it to the user. This historical comparison enables the health coach to detect trends in the user's metabolic health and to adjust recommendations to reflect improving or deteriorating conditions.

The health coach requests, with a voice output, an additional health information input from the user, receives the additional health information input from the user as a voice input, and modifies the personalized health intervention information based on the additional health information input before outputting the personalized health intervention information to the user. This interactive data gathering enables the health coach to collect clarifying information that improves the accuracy and relevance of the generated health intervention information.

The health coach receives, after outputting the personalized health intervention information to the user, an additional input from the user, modifies the personalized health intervention information based on the additional input from the user, and outputs the modified personalized health intervention information to the user. For example, if the user's biomarkers improve or if the user reports experiencing side effects, the health coach modifies the personalized health intervention information based on the additional input.

The health coach predicts a risk of an adverse event such as a hyperglycemic episode or a high HbA1c reading in a future time period. The health coach modifies the personalized health intervention information to include a preventative recommendation based on the predicted risk of an adverse event. The predictive analysis uses the biometric data trends, the user's historical health data, and the large language model's health-specific training to generate forward-looking assessments and proactive warnings.

The health coach performs additional steps including requesting additional inputs from the user, generating a second personalized health intervention information based on the additional inputs and any updated biometric data, and outputting the second personalized health intervention information to the user. This enables multi-turn health coaching conversations where the health coach progressively refines its understanding of the user's condition and provides increasingly targeted intervention recommendations.

Although the disclosed embodiments have been illustrated and described with respect to one or more implementations, equivalent alterations and modifications will occur or be known to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

While various embodiments of the present disclosure have been described above, it should be understood that they have been presented by way of example only, and not limitation. Numerous changes to the disclosed embodiments can be made in accordance with the disclosure herein, without departing from the spirit or scope of the disclosure. Thus, the breadth and scope of the present disclosure should not be limited by any of the above described embodiments. Rather, the scope of the disclosure should be defined in accordance with the following claims and their equivalents.

What is claimed is:

1. A method comprising:
receiving, from a user, a voice input through a voice interface;
interpreting the voice input with natural language processing performed by an artificial intelligence model that includes a large language model to determine a meaning of the voice input, the large language model including whether the voice input requests personalized health intervention information;
receiving a glucose level from a continuous glucose monitor;
receiving an insulin level retrieved from a database;
calculating, with a processor, a Homeostatic Model Assessment of Insulin Resistance score from the glucose level and the insulin level;
comparing the Homeostatic Model Assessment of Insulin Resistance score to historical Homeostatic Model Assessment of Insulin Resistance scores associated with the user to determine whether the Homeostatic Model Assessment of Insulin Resistance score has changed over time;
identifying, based on the Homeostatic Model Assessment of Insulin Resistance score and the comparison to historical scores, a predicted risk of a hyperglycemic episode;
generating, with the processor using the artificial intelligence model, a preventative health recommendation based on the predicted risk of the hyperglycemic episode and modified based on whether the Homeostatic Model Assessment of Insulin Resistance score has changed over time;
requesting, with a voice output through the voice interface, additional health information from the user;
receiving the additional health information from the user as a voice input;
modifying the preventative health recommendation based on the additional health information; and
outputting the modified preventative health recommendation to the user.

2. The method of claim 1, further comprising performing a prosody analysis on the voice input to detect emotional cues, wherein the preventative health recommendation is provided as an output in the form of a voice output that is generated through text-to-speech synthesis with emotion modulation.

3. The method of claim 1, further comprising receiving real-time biometric data from at least one of a wearable health monitoring device, a fitness tracker, or a smart medical device, wherein the real-time biometric data is used in generating the preventative health recommendation.

4. The method of claim 1, wherein the Homeostatic Model Assessment of Insulin Resistance score is used as an insulin resistance index to calculate a metabolic health index, the metabolic health index including at least the Homeostatic Model Assessment of Insulin Resistance score calculated from the glucose level and the insulin level.

5. The method of claim 4, further comprising:
comparing the metabolic health index to historical metabolic health index data associated with the user to determine if the metabolic health index has changed over time; and
modifying the preventative health recommendation based on whether the metabolic health index has changed over time.

6. The method of claim 1, wherein identifying the predicted risk of the hyperglycemic episode includes comparing the Homeostatic Model Assessment of Insulin Resistance score to historical Homeostatic Model Assessment of Insulin Resistance scores to identify a predicted risk of a hyperglycemic episode, the preventative health recommendation including a dietary or lifestyle modification based on the predicted risk.

7. The method of claim 1, further comprising:
receiving, after outputting the modified preventative health recommendation, a second additional input from the user;
generating a second preventative health recommendation based on the second additional input and any updated biometric data; and
outputting the second preventative health recommendation to the user.

8. The method of claim 1, further comprising:
receiving a plurality of physiological measurements from a plurality of sensors, the plurality of physiological measurements including at least two of a glucose level, an insulin level, a heart rate, heart rate variability, blood oxygen saturation, or a glycated hemoglobin value; and
generating the preventative health recommendation further based on the plurality of physiological measurements.

9. The method of claim 1, further comprising:
storing, in a user data storage, user data comprising conversation records, recorded emotional states, biometric data history, and user preferences;
using the stored user data as contextual memory to interpret the voice input and generate the preventative health recommendation across multiple interaction sessions; and
interpreting ambiguous voice inputs based on the contextual memory of prior conversations with the user.

10. The method of claim 1, further comprising:
receiving a lab test result automatically retrieved from a database, the lab test result including data obtained from at least one of laboratory, metabolic, hormonal, or microbiome-related tests, and
using the lab test result in generating the preventative health recommendation.

11. The method of claim 1, wherein the artificial intelligence model or the voice interface introduces controlled imperfections into outputs including the preventative health recommendation, the controlled imperfections including at least one of inserting a hesitation pause, generating a self-correction phrase, generating an admission of uncertainty, or varying response time, wherein the voice interface maintains a consistent tone across multiple interaction sessions through style transfer techniques applied to the outputs.

12. The method of claim 1, wherein the voice interface includes conversation steering rules that reject or modify outputs that deviate from predefined health-related topics, the voice interface further including a turn-taking mechanism that delays response generation until the user has finished providing input.

13. A non-transitory computer-readable medium storing instructions that, when executed by one or more processors of a device, cause the device to:
receive a user input;
interpret the user input with natural language processing performed by an artificial intelligence model that includes a large language model to determine a meaning of the user input, including whether the user input requests personalized health intervention information;
receive a glucose level from a continuous glucose monitor;
receive an insulin level retrieved from a database; calculate a metabolic health index from the glucose level and the insulin level, the metabolic health index including at least an insulin resistance index calculated from the glucose level and the insulin level;
analyze the glucose level, the insulin level, and the metabolic health index with the artificial intelligence model;
identify, based on the metabolic health index and the meaning of the user input, a physiological trend of the user;
compare the metabolic health index to historical metabolic health index data associated with the user to determine whether the metabolic health index has changed over time;
generate, using the artificial intelligence model, personalized health intervention information relating to the identified physiological trend of the user and modified based on whether the metabolic health index has changed over time; and
output the personalized health intervention information to the user.

14. The non-transitory computer-readable medium of claim 13, wherein the insulin resistance index is a Homeostatic Model Assessment of Insulin Resistance score, the glucose level is received from a continuous glucose monitor, the insulin level is retrieved from a database, and the personalized health intervention information includes a preventative recommendation addressing a predicted adverse metabolic health event.

15. The non-transitory computer-readable medium of claim 13, wherein the instructions further cause the device to:
store user data that includes conversation records, recorded emotional states, biometric data history, and user preferences; and
use the stored user data as contextual memory to interpret user inputs and generate personalized health intervention information across multiple interaction sessions.

16. The non-transitory computer-readable medium of claim 13, wherein identifying the physiological trend or condition includes comparing biometric data trends to historical metabolic health index data associated with the user to identify a predicted risk of a hyperglycemic episode, and the personalized health intervention information includes a warning and a preventative recommendation addressing the predicted adverse metabolic health event.

17. A health coach system comprising:
a voice interface configured to receive a user input including at least one of a spoken input through a microphone, a typed input through a keyboard, or a gestural input through a camera, the voice interface including computer vision models configured to (a) recognize and interpret gestures and facial expressions, and (b) to process the user input using natural language processing, wherein when the user input includes the spoken input the voice interface performs prosody analysis on the spoken input to detect emotional cues;
one or more sensors including a continuous glucose monitor configured to provide a glucose level to the health coach system;
one or more data sources configured to provide an insulin level retrieved from a database;
one or more processors configured to calculate a Homeostatic Model Assessment of Insulin Resistance score from the glucose level and the insulin level, the one or more processors further being configured to compare the Homeostatic Model Assessment of Insulin Resistance score to historical Homeostatic Model Assessment of Insulin Resistance scores associated with the user to determine whether the score has changed over time;
a large language model configured to receive the processed user input, the biometric data, and the calculated Homeostatic Model Assessment of Insulin Resistance score, the large language model being further configured to analyze the biometric data and identify a physiological trend or condition of the user, the large language model being further configured to generate personalized health intervention information relating to the identified physiological trend or condition and modified based on whether the Homeostatic Model Assessment of Insulin Resistance score has changed over time;
a user data storage configured to store conversation records, recorded emotional states, biometric data history, and user preferences as a persistent user profile; and
an output interface configured to output the personalized health intervention information to the user through at least one of a speaker or a screen;
wherein the health coach system uses the persistent user profile as contextual memory to interpret user inputs and generate personalized health intervention information across multiple interaction sessions.

18. The system of claim 17, wherein the voice interface combines a plurality of inputs from a plurality of input devices into a combined input provided to the large language model, and wherein the plurality of input devices includes at least one biometric sensor configured to provide real-time physiological measurements to the health coach system.

19. The system of claim 17, wherein the one or more sensors include a continuous glucose monitor, and wherein the one or more processors calculate a Homeostatic Model Assessment of Insulin Resistance score from glucose and insulin data, the health coach system being configured to predict a risk of a hyperglycemic episode, based on biometric data trends and historical user data, and to generate a preventative recommendation.

20. The system of claim 17, wherein the voice interface maintains a consistent tone across multiple interactions through style transfer techniques applied to outputs, introduces controlled imperfections into outputs that include at least one of inserting a hesitation pause, generating a self-correction phrase, or generating an admission of uncertainty, and includes conversation steering rules that reject or modify outputs that deviate from predefined health-related topics.
